(19) United States
(12) Patent Application Publication
(10) Pub. No.: US 2013/0226306 A1
(43) Pub. Date: Aug. 29, 2013

(54) POLYCARBONATE URETHANE JOINT IMPLANT

(71) Applicant: Sanjiv Naidu, Lititz, PA (US)

(72) Inventor: Sanjiv Naidu, Lititz, PA (US)

(73) Assignee: REMI SCIENCES, INC., Lititz, PA (US)

(21) Appl. No.: 13/408,574

(22) Filed: Feb. 29, 2012

(51) Int. Cl. A61F 2/42

(52) U.S. Cl. 623/21.12

(57) ABSTRACT

A compressive force and compressive-shear force joint implant including a head defining at least one wear contact surface. At least the at least one wear contact surface is manufactured from a polycarbonate urethane material. The implant may further include a stem extending from the head opposite of the wear contact surface. The head may also be configured to define a second wear contact surface distinct from the first wear contact surface.

### FIELD OF THE INVENTION

This relates to the field of medical devices and more particular to a compressive-shear wear joint replacement.

### BACKGROUND OF THE INVENTION

Arthritis of the thumb basal joint (or alternatively refered to as the thumb carpometacarpal (CMC) joint) or the trapeziometacarpal joint (TMJ) joint is a disabling disorder of the thumb axis. Similarly, arthritis of the metatarsophalangeal joint (MTPJ) is a disabling disorder of the toe axis. Similarly, arthritis of the tarsometatarsal joints (TMT) is a disabling disorder of the feet. Similarly, arthritis and instability of the radiocapitellar joint is a disabling disorder of the elbow joint.

Since the early 1960s, various solutions have been introduced for reconstruction of these joints to try to alievate the pain and discomfort. Silicone replacement arthroplasty of the thumb CMC was first advocated by Swanson in the early 1960s, however, such silicone joint replacements have essentially fell out of favor mainly because of the complications associated with wear of the silicone implant, and silicone synovitis. Silicone synovitis is essentially a recurrence of pain, swelling, and instability at the site of the original silicone replacement arthroplasty. It is characterized by bony destruction, and soft tissue swelling and inflammation. FIG. 1 illustrates exemplary prior art silicone joint implants 10, 10′, with the implant 10 illustrating a condition prior to use and the implant 10′ showing wear to a head portion 12′ of the implant 10′ after use. Similarly, FIG. 2 shows a silicone test implant 20 showing fragmentation wear after a wear test as described below.

Another problem associated with silicone implants is silicone elastomer transfer wear which causes a spackling effect against the bone wherein pores of the bone are filled with the silicone. FIG. 3 shows a scanning electron microscope picture of the surface of an artificial bone 30 counter face used in the wear test as described below. As seen therein, after repeated contact between the test implant 20 against the artificial bone 30, a significant amount of silicone material 34 transferred to the artificial bone 30 and filled the pores 32 and formed ridges 36.

Subsequently various metallic, ceramic, absorbable polymeric, and pyro carbon implants have been introduced to serve either as spacers or hemiarthroplasty in order to provide for pain relief at the CMC, TMJ, MTPJ and radiocapitellar joints.

Biomechanically, the prior art implants are either too stiff, or too soft to provide for a durable arthroplasty. For example, the stiffness of the trapezium generally is essentially similar to that of the scaphoid at approximately 150 Megapascals. The silicone implants initially advocated in the 1960s display a stiffness of less than 4 megapascals in vivo, where as the titanium implants are in general more than 100 Gigapascals. The cobalt chrome trapezial implants display a high stiffness at 200 GigaPascals while the zirconia ceramic implants are even stiffer at approximately 400 GigaPascals. The more recent pyrocarbon introduction is an attempt to use materials which are less stiff, however, the pyrocarbon stiffness nevertheless approaches that of cortical bone at approximately 15-20 GigaPascals (3 orders of magnitude more stiff than the native trapezium). Accordingly, these materials do not provide a biomechanically appropriate implant.

### BRIEF SUMMARY OF THE INVENTION

Looking at the CMC, for example, the ideal material for joint replacement arthroplasty would not only be mechanically and materially less stiff than the trapezium to provide for a stable spacer to prevent collapse of the thumb, but also would be less in stiffness to that of the cortico-cancellus bone of the thumb metacarpal medullary shaft in order to prevent thumb metacarpal subsidence over the implant. In addition, an ideal material would have superior wear qualities so that microscopic wear particles would not create polymeric synovitis. In short, material that is slightly stiffer than silicone elastomer yet resistant to in vivo degradation with superior wear properties would be an ideal candidate to serve as a sound CMC, TMJ, MTPJ or radiocapitellar joint implant.

The inventor has recognized that polycarbonate urethanes (PCU), which are a class of thermoplastic polyurethanes (TPU), allow for desired elastomeric properties to be maintained in vivo, while at the same time provide for adequate protection against environmental stress cracking and breakdown in vivo.

The present invention provides in at least one embodiment a compressive force and compressive-shear force joint implant including a head defining a wear contact surface and a stem extending from the head opposite of the wear contact surface. At least the wear contact surface is manufactured from a polycarbonate urethane material.

In at least one embodiment, the present invention provides a compressive force and compressive-shear force joint implant including a head defining at least two wear contact surfaces with at least the wear contact surfaces manufactured from a polycarbonate urethane material.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a photograph of prior art silicone implants, with one of the implants shown prior to use and the other shown after use in a patient.

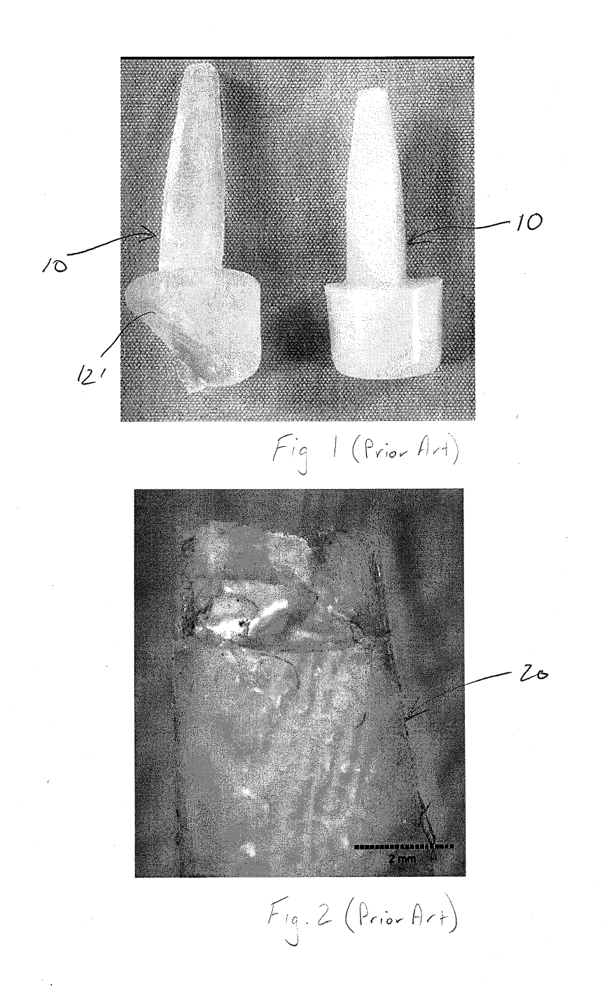

FIG. 2 is a photograph of test silicone implant after being subjected to a wear test.

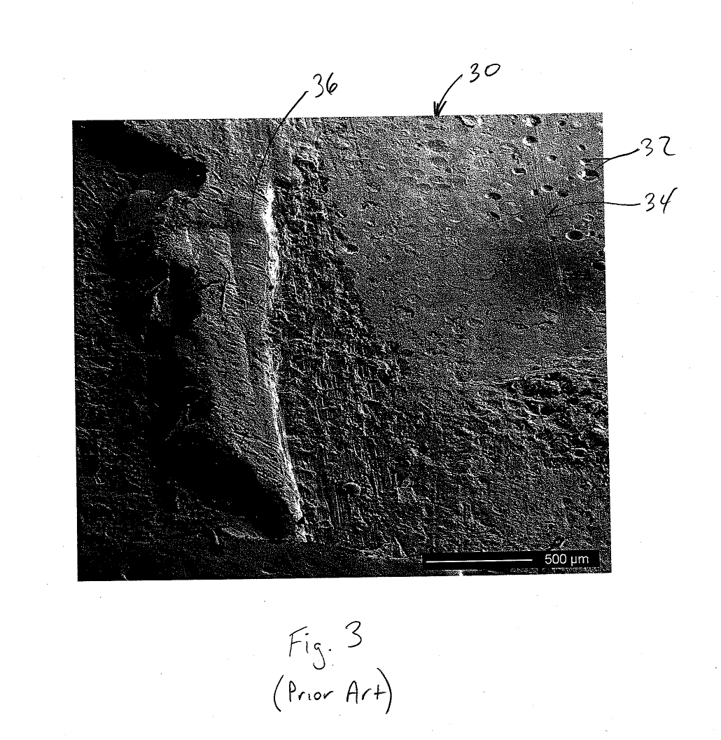

FIG. 3 is a scanning electron microscope picture of the surface of an artificial bone counter face used with the test silicone implant in the wear test.

FIG. 4 is a schematic drawing of an exemplary implant of the invention positioned in a CMC arthroplasty.

FIG. 5 is a schematic drawing of an exemplary implant of the invention positioned in a TMJ arthroplasty.

FIG. 6 is a schematic drawing of exemplary implants of the invention positioned in a TMJ arthroplasty.

FIG. 7 is a schematic drawing of an exemplary implant of the invention positioned in a MTPJ arthroplasty.

FIG. 8 is a schematic drawing of an exemplary implant of the invention positioned in a radiocapitellar joint arthroplasty.

FIG. 9 is an isometric view of an implant in accordance with a first exemplary embodiment of the invention.

FIG. 10 is a cross-sectional view of an implant in accordance with another exemplary embodiment of the invention.

FIGS. 11-19 are isometric views of implants in accordance with various other exemplary embodiments of the invention.

FIG. 20 is a schematic drawing of another exemplary implant of the invention positioned in a CMC arthroplasty.

FIG. 21 is a schematic drawing of anonther exemplary implant of the invention positioned in a CMC arthroplasty.

FIG. 22 is a schematic view of a wear test assembly utilized to test the wear characteristics of an implant in accordance with an exemplary embodiment of the invention versus a prior art silicone test implant.

FIG. 23 is a scanning electron microscope picture of the surface of an artificial bone counter face used with the implant in accordance with an exemplary embodiment of the invention in the wear test.

FIG. 24 is a graph illustrating a dynamic mechanical analysis of the implant in accordance with an exemplary embodiment of the invention.

FIG. 25 is a graph illustrating a dynamic mechanical analysis of a prior art silicone test implant.

FIG. 26 is a schematic view of a compression test assembly utilized to test the compression fatigue characteristics of an implant in accordance with an exemplary embodiment of the invention versus a prior art silicone test implant.

FIGS. 27-31 are graphs illustrating the cyclic compressive deformation of an implant in accordance with an exemplary embodiment of the invention under various testing conditions.

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

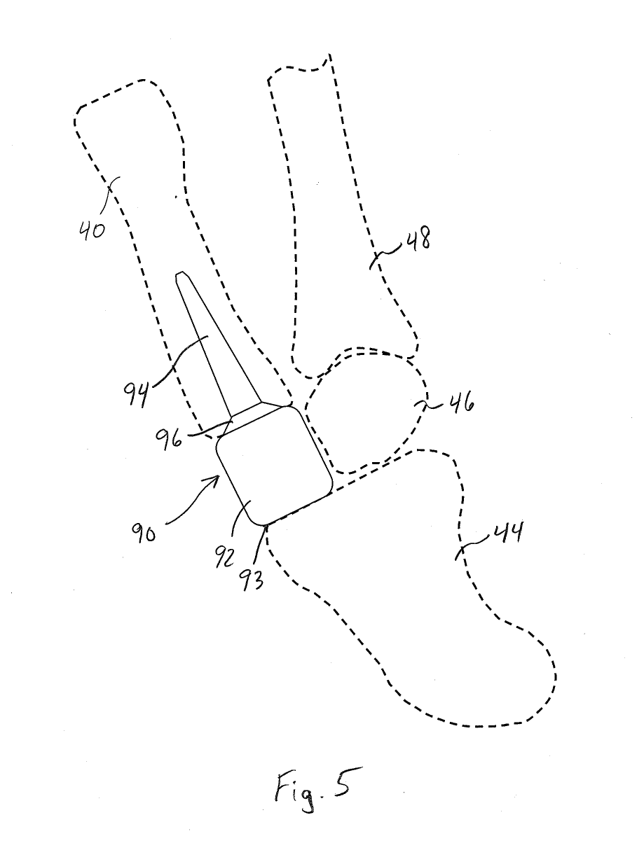

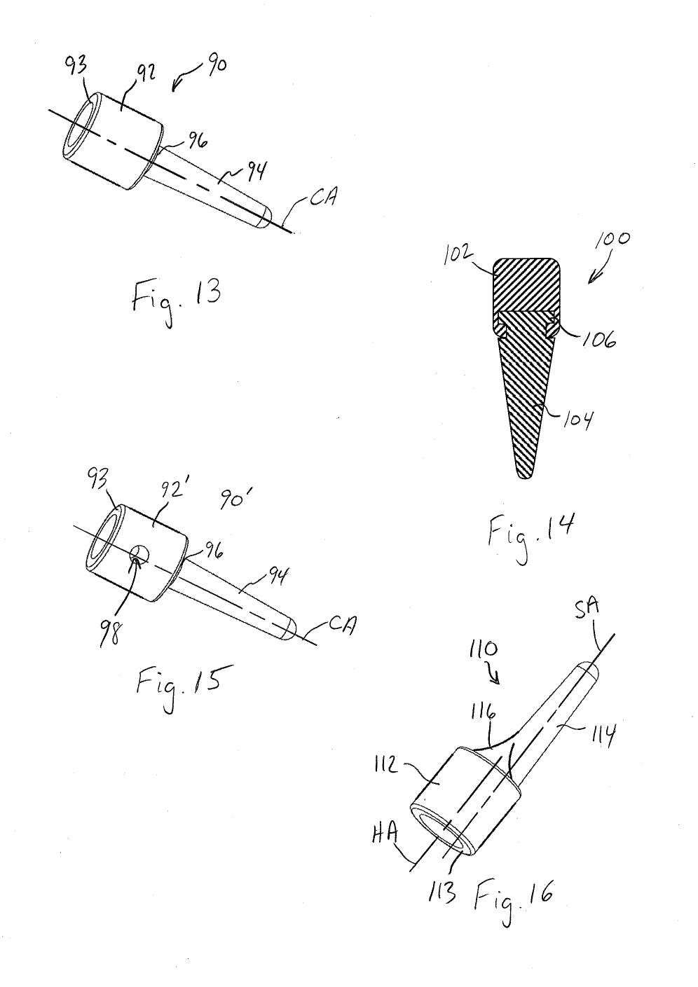

Referring to FIG. 4, a CMC arthroplasty is illustrated with an exemplary implant 50 positioned between the thumb metacarpal 40 and the remaining portion of the trapezium 42. For context, the scaphoid 44, trapezoid 46 and the next metacarpal 48 are illustrated. With reference also to FIG. 9, the exemplary implant 50 includes a cylindrical head 52 connected to a stem 54 via a collar 56. The head 52 defines a wear contact surface 53 which is opposite the stem 54. Upon implantation in a known manner, the stem 54 extends into a bore formed in the metacarpal 40 and the wear contact surface 53 bears against the portion of the trapezium 42 in compressive contact. The interaction between the wear contact surface 53 and the portion of the trapezium 42 allows for the normal multidirectional movement of the thumb. As used herein, the term wear contact surface refers to a surface of the implant configured to be placed in compressive contact with an opposed structure, e.g. bone or another implant member, with relative movement between the wear contact surface and the opposed structure.

In the present embodiment, the head 52, including the wear contact surface 53, the stem 54 and the collar 56 are formed as a unitary structure of PCU material. While the present embodiment is illustrated as a unitary structure, the invention is not limited to such. For example, the implant 100 illustrated in FIG. 14 includes a head 102 with a wear contact surface 103 and a separate stem 104 with a locking collar 106. The stem 104 and collar 106 may be manufactured from, for example, a biocompatible metal or ceramic material while the head 102 is manufactured from PCU material. The head 102 may be overmolded about the collar 106, snap-fit to the collar 106 or otherwise connected thereto.

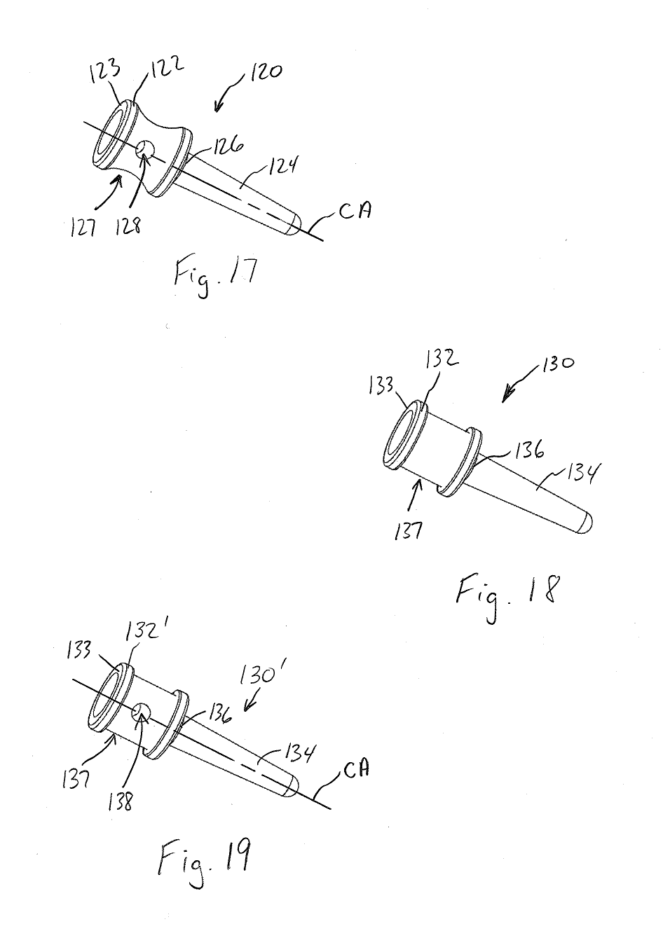

In the implant 50 of FIG. 9, the head 52 and the stem 54 are co-axial with a central axis CA extending through the center of each, however, the invention is not limited to such a configuration. FIG. 10 illustrates an implant 60 with a head 62 defining a wear contact surface 63 on one side and a stem 64 extending from the opposite side of the head 62. The stem 64 has an axis SA which is offset from the axis HA of the head 62. The collar 66 is preferably configured to accommodate the offset. The offset allows the implant 60 to compensate for bone misalignments or allow use in alternative structures. Otherwise the implant 60 is as described with respect to implant 50 and includes a head 62 and wear contact surface 63 manufactured from PCU material. The implant 60 may be a unitary structure or a multipart structure as described above.

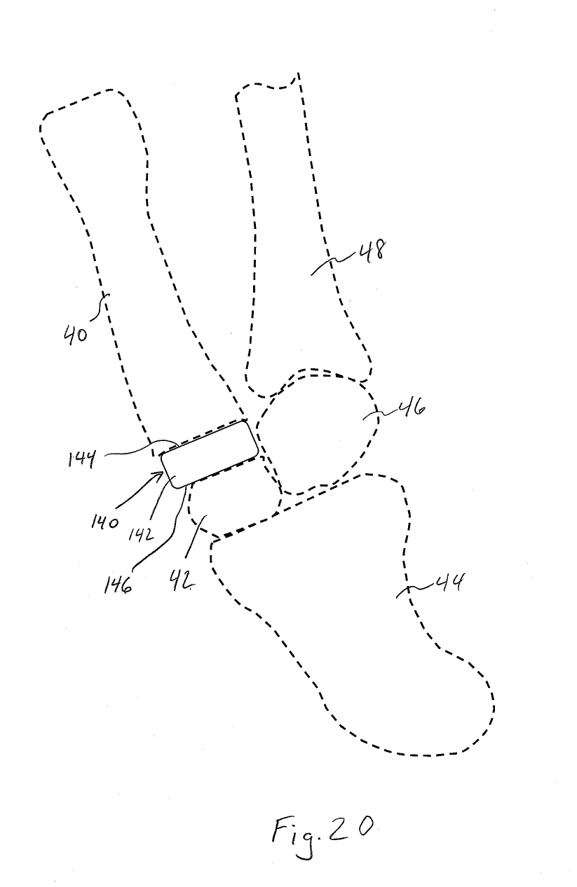

The implant 50 of FIG. 9 has a planar wear contact surface 53 which is substantially perpendicular to the central axis CA, however, the invention is not limited to such a configuration. FIGS. 11 and 12 illustrate implants 70 and 80 each having a head 72, 82 defining a hemispherical wear contact surface 73, 83. A stem 74, 84 extends from the opposite side of the head 72, 74 and is interconnected via a collar 76, 86. The stem 74 and head 72 of the implant 70 are co-axial while the stem 84 and head 82 of the implant 80 are offset. Otherwise the implants 7, 800 are as described with respect to implant 50 and include a head 72, 82 and wear contact surface 73, 83 manufactured from PCU material. The implants 70, 80 may each have a unitary structure or a multipart structure as described above.

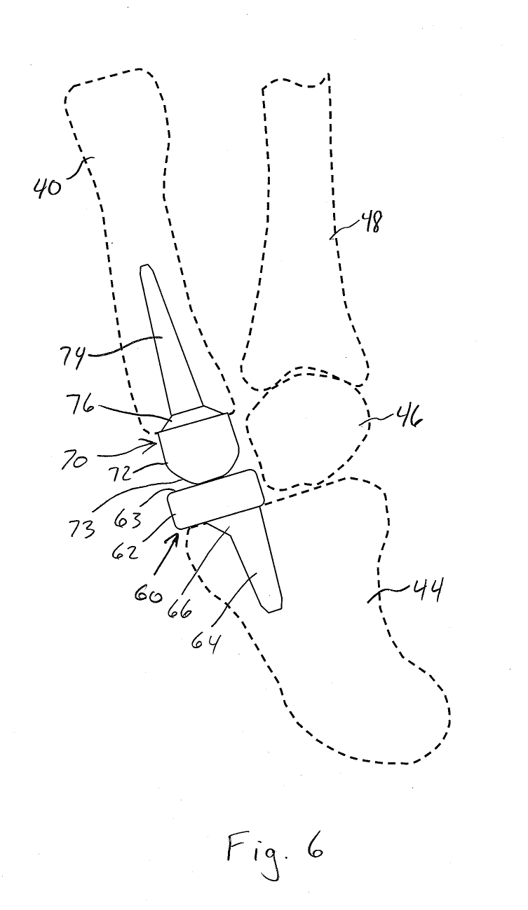

FIG. 5 illustrates a TMJ arthroplasty with the trapezium completely removed and an exemplary implant 90 positioned between the thumb metacarpal 40 and the scaphoid 44. The implant 90 is similar to the implant 50 and includes a cylindrical head 92 connected to a stem 94 via a collar 96. The head 92 defines a wear contact surface 93 which is opposite the stem 94. Upon implantation in a known manner, the stem 94 extends into a bore formed in the metacarpal 40 and the wear contact surface 93 bears against the scaphoid 44. It is noted that the head 92 is longer than the head 52 to compensate for the larger distance between the metacarpal 40 and the scaphoid 44. The interaction between the wear contact surface 93 and the scaphoid 44 allows for the normal multidirectional movement of the thumb. The implant 90 is similar to implant 50 and includes a head 92 and wear contact surface 93 manufactured from PCU material. The implant 90 may be a unitary structure or a multipart structure as described above and illustrated in FIG. 14.

FIG. 15 illustrates an implant 90′ substantially the same as the implant 90, however the implant 90′ includes a cross bore 98 extending through the head 92′ substantially perpendicular to the central axis CA. The cross bore 98 provides for tendon passage to secure the implant 90′. In all other respects, the implant 90′ is the same as the implant 90.

FIGS. 16-19 illustrate alternative exemplary implants 110, 120, 130 and 130′ which are similar to the implant 90. The implant 110 of FIG. 16 includes a cylindrical head 112 with a wear contact surface 113, a stem 114 and a collar 116. The implant 110 differs from implant 90 only in that the axis HA of the head 112 is offset from the axis SA of the stem 114.

The implant 120 of FIG. 17 includes a cylindrical head 122 with a wear contact surface 123, a stem 124 and a collar 126. The implant 120 differs from implant 90 in that the head 122 includes an annular convex groove 127 and a cross bore 128 similar to implant 90′. The groove 127 and the cross bore 128 facilitate placement and securement of one or more tendons to the implant 120.

The implants 130, 130′ of FIGS. 18 and 19 include a cylindrical head 132, 132′ with a wear contact surface 133, a stem 134 and a collar 136. The implants 130, 130′ differ from implant 90 in that the head 132, 132′ includes an annular rectangular groove 137 and the head 132′ of implant 130′ further includes a cross bore 138.

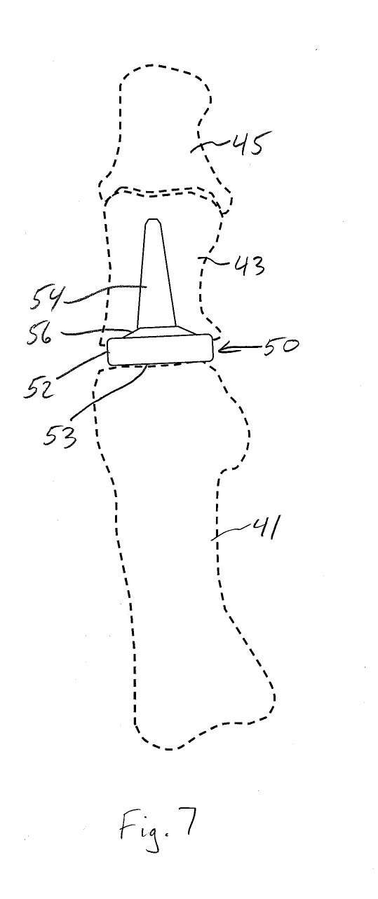

Similar to FIG. 5, FIG. 6 illustrates a TMJ arthroplasty with the trapezium completely removed, however, a pair of implants 60 and 70 are positioned between the thumb metacarpal 40 and the scaphoid 44. The stem 74 of implant 70 is fixed in the metacarpal 40 while the stem 64 of implant 60 is fixed in the scaphoid 44. The wear contact surfaces 63, 73 of the implants 60, 70 face one another and are in compressive contact. The interaction between the wear contact surfaces 63 and 73 allows for the normal multidirectional movement of the thumb.

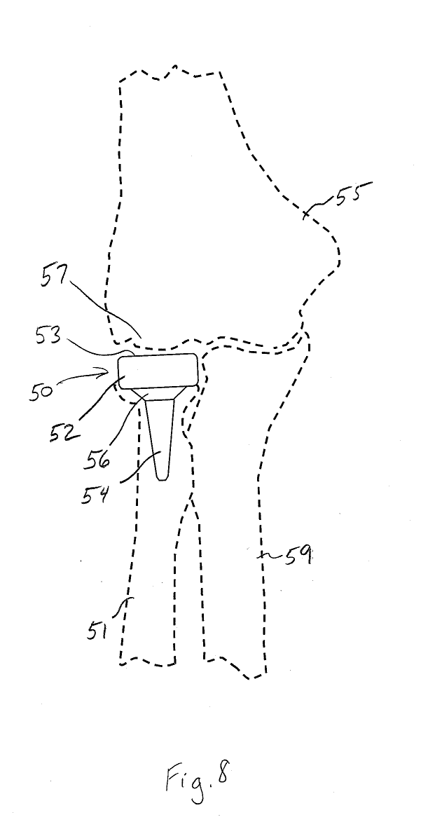

Referring to FIG. 7, an MTPJ arthroplasty is illustrated with an exemplary implant 50 positioned between the toe metatarsal 41 and the remaining portion of the proximal phalange 43. For context, the distal phalange 45 is illustrated. Upon implantation in a known manner, the stem 54 extends into a bore formed in the proximal phalange 43 and the wear contact surface 53 bears against the metatarsal 41 in compressive contact. The interaction between the wear contact surface 53 and the metatarsal 41 allows for the normal multidirectional movement of the toe. While illustrated with respect to the MTPJ, the implant 50 may similarly be positioned between the metatarsal 41 and the cuneiform to provide TMT joint arthroplasty.

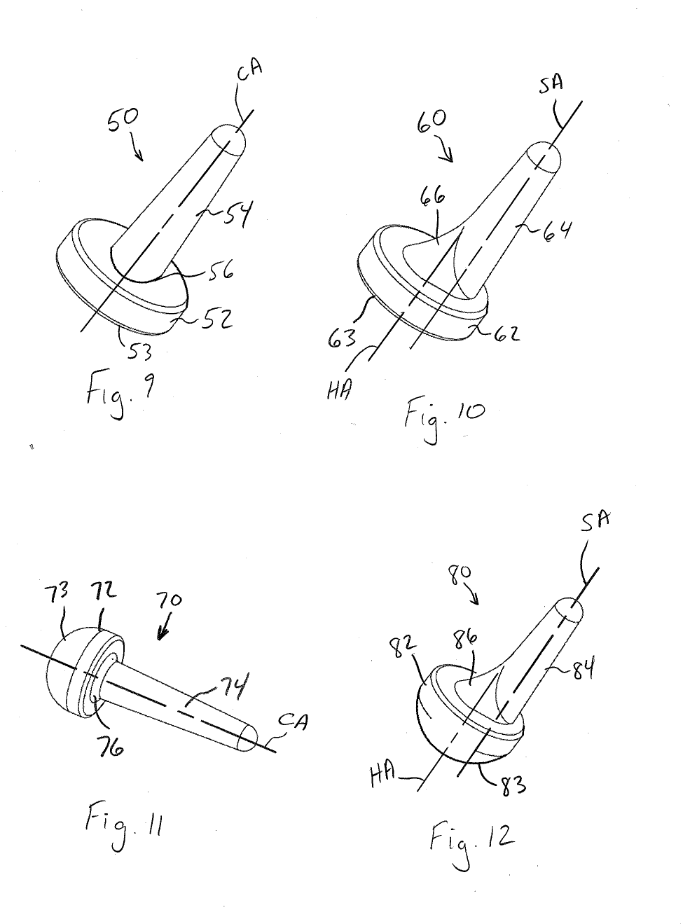

Referring to FIG. 8, a radiocapitellar joint arthroplasty is illustrated with an exemplary implant 50 positioned between the radius 51 and the capitulum 57 of the humerus 55. For context, the ulna 59 is illustrated. Upon implantation in a known manner, the stem 54 extends into a bore formed in the radius 51 and the wear contact surface 53 bears against the capitulum 57 in compressive contact. The interaction between the wear contact surface 53 and the capitulum 57 allows for the normal multidirectional movement of the elbow.

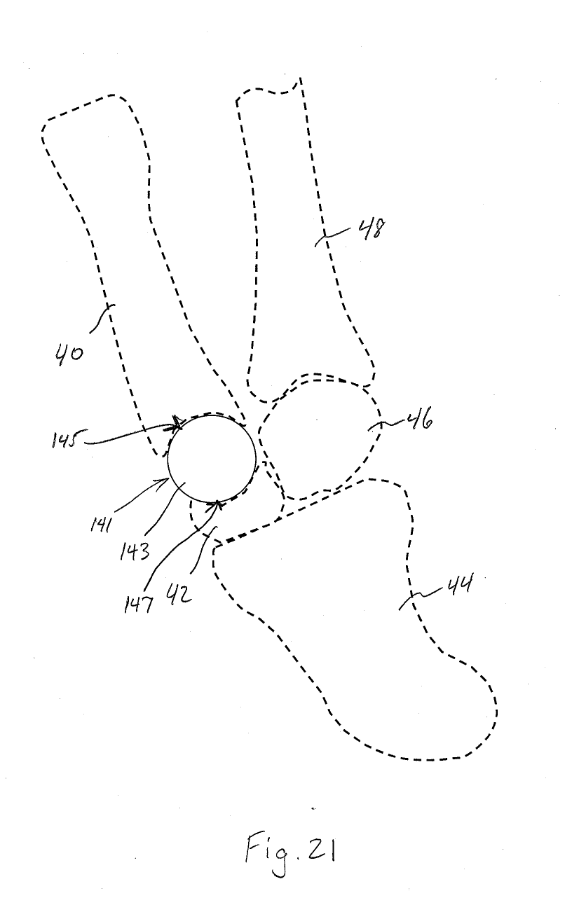

Referring to FIG. 20, a CMC arthroplasty is illustrated with another exemplary implant 140 positioned between the thumb metacarpal 40 and the remaining portion of the trapezium 42. In the present embodiment, the exemplary implant 140 includes a cylindrical head 142 which defines opposed wear contact surfaces 144 and 146. The implant 140 does not include a stem and is configured to be positioned between and held in place by the existing bone structures 40 and 42. The contact ends of the bone structures 40 and 42 may be shaped prior to positioning of the implant 140 such that the implant 140 is retained within a concave configuration of one or both bone structures 40, 42. Upon implantation, the wear contact surface 144 bears against the portion of the metacarpal 40 in compressive contact and the wear contact surface 146 bears against the portion of the trapezium 42 in compressive contact. The interaction between the wear contact surfaces 144 and 146 and the metacarpal 40 and the portion of the trapezium 42, respectively, allows for the normal multidirectional movement of the thumb. The head 142 may include a cross bore as described in conjunction with some of the prior embodiments. In a preferred embodiment, the entire head 142, including the wear contact surfaces 144 and 146, is manufactured from PCU material, however, the implant 140 may have other configurations, for example, a composite structure wherein only the wear contact surfaces 144 and 146 are manufactured from PCU material.

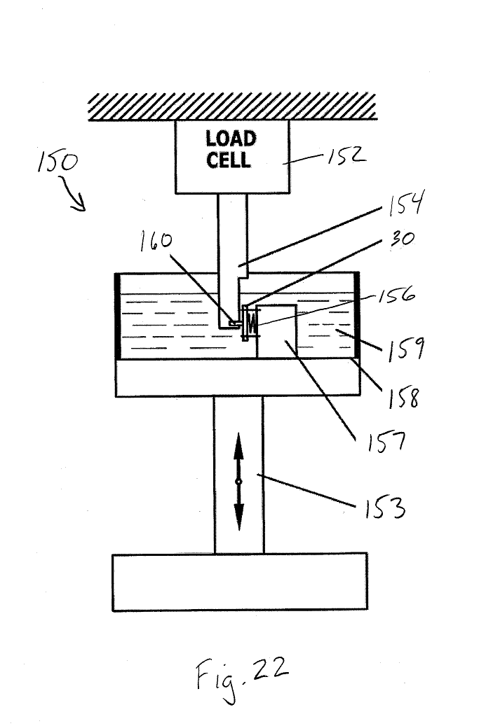

Referring to FIG. 21, a CMC arthroplasty is illustrated with another exemplary implant 141 positioned between the thumb metacarpal 40 and the remaining portion of the trapezium 42. In the present embodiment, the exemplary implant 141 includes a spherical head 143 which defines opposed wear contact surfaces 145 and 147. The implant 141 does not include a stem and is configured to be positioned between and held in place by the existing bone structures 40 and 42. The contact ends of the bone structures 40 and 42 may be shaped prior to positioning of the implant 141 such that the implant 141 is retained within a concave configuration of one or both bone structures 40, 42. Upon implantation, the wear contact surface 145 bears against the portion of the metacarpal 40 in compressive contact and the wear contact surface 147 bears against the portion of the trapezium 42 in compressive contact. The interaction between the wear contact surfaces 145 and 147 and the metacarpal 40 and the portion of the trapezium 42, respectively, allows for the normal multidirectional movement of the thumb. The head 143 may include a cross bore as described in conjunction with some of the prior embodiments. In a preferred embodiment, the entire head 143, including the wear contact surfaces 145 and 147, is manufactured from PCU material, however, the implant 141 may have other configurations, for example, a composite structure wherein only the wear contact surfaces 145 and 147 are manufactured from PCU material.

While the present invention is described herein in relation to CMC, TMJ, MTPJ and radiocapitellar joint arthroplasty, the invention is not limited to such. Implants in accordance with the invention may be utilized in other applications wherein the implant wear contact surface is subject to compressive contact. Additionally, while various embodiments of the implant are described herein, the invention is not limited to such. The implants may have various configurations with a head having a wear contact surface manufactured from PCU material. As explained in more detail below, the use of such PCU material provides unexpected favorable results for a compressive implant having a head with a wear surface on one side and a stem extending from the opposite side. Such an implant meets the need for a reliable implant that has existed since the 1960s.

To confirm the viability of the implants of the present invention, a wear test was performed on an exemplary PCU implant and a prior art silicone implant. In general, post reconstruction of the thumb basal joint, the maximum key pinch strength obtained is approximately 5±2.5 kilograms; activities of daily living require a pinch force no more than 2 kilograms. Therefore a normal force of 8 pounds was chosen to be applied to the prosthetic stem against synthetic bone #40 (Pacific research labs) to study wear characteristics.

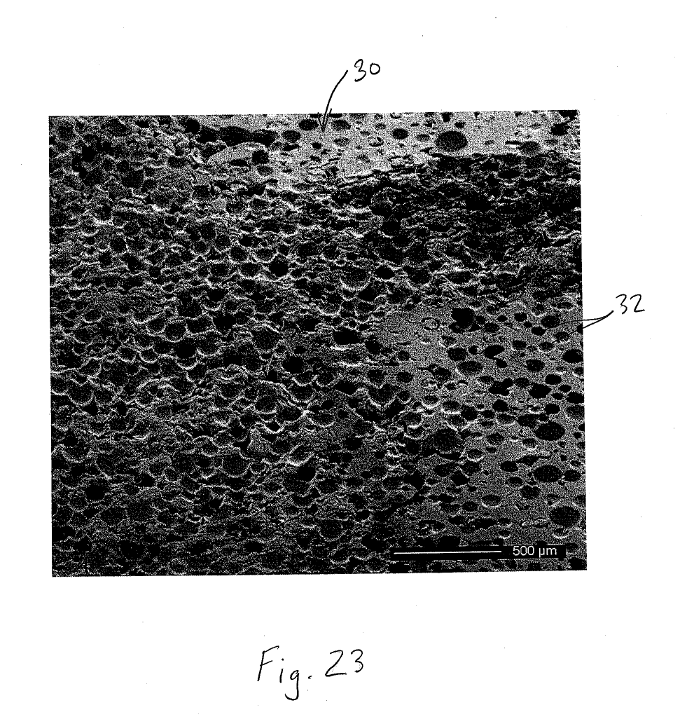

Tests were performed on both silicone implants from Wright medical technology (flexspan) and the PCU implants of the present invention. Testing was performed utilizing a wear test assembly 150 as illustrated in FIG. 22. The specimens 160 were secured in a stainless steel rod 154 suspended from a load cell 152 over a fluid chamber 158. The chamber 158 was filled with saline at 37° C. to simulate in vivo conditions. Each specimen 160 was equilibrated in the saline 159 for two days before the test. An artificial bone sample 30 was supported by a spring 156 extending from a support member 157. The spring 156 urged the artificial bone sample 30 into contact with the sample 160 with the desired 8 pound normal force. An actuator 153 oscillated the artificial bone sample 30 relative to the specimen 160 to conduct the test. After 221,000 cycles, weight loss from the samples were recorded.

Table 1 below provides a summary of the weight loss during the wear test results while Table 2 shows the normalized percentage of weight loss results of the test. As can be seen, there was significantly more weight loss in the silicone group when compared to the PCU implant group.

TABLE 1
Wear Test Summary

| | |
|---|---|
| Flexspan (Wright) | PCU Implant |

Weight Loss (mg)

| | | | |
|---|---|---|---|
| Sample | 1 | 26.0 | 3.3 |
| Number | 2 | 10.6 | 3.8 |
| | 3 | 15.2 | 3.0 |
| | 4 | 17.5 | 6.5 |
| | 5 | 17.7 | 9.3 |
| | 6 | 16.9 | 4.6 |

| | | |
|---|---|---|
| Mean | 17.3 | 5.1 |
| Std. Dev. | 5.0 | 2.4 |

TABLE 2
Wear Test Summary

| | | Flexspan (Wright) | | | | PCU Implant | | | |
|---|---|---|---|---|---|---|---|---|---|
| | | Weight | Weight | | | Weight | Weight | | |
| | | Before | After | Weight | Coef. | Before | After | Weight | Coef. |
| | | Test | Test | Loss | Of | Test | Test | Loss | Of |
| | | (mg) | (mg) | (%) | Friction | (mg) | (mg) | (%) | Friction |
| Sample | 1 | 224.3 | 198.3 | 11.59 | 0.41 | 190.0 | 186.7 | 1.74 | 0.66 |
| Number | 2 | 196.7 | 186.1 | 5.39 | 0.45 | 152.6 | 148.8 | 2.49 | 0.70 |
| | 3 | 173.7 | 158.5 | 8.75 | 0.42 | 177.7 | 174.7 | 1.69 | 0.68 |
| | 4 | 221.2 | 211.9 | 9.3 | 0.43 | 169.2 | 165.0 | 2.48 | 0.60 |
| | 5 | 183.5 | 165.3 | 9.92 | 0.45 | 196.8 | 192.9 | 1.96 | 0.583 |
| | 6 | 181.6 | 175.6 | 5.95 | 0.43 | 200.9 | 196.3 | 2.30 | 0.68 |

| | | | | | | | | |
|---|---|---|---|---|---|---|---|---|
| Mean | 196.83 | 182.62 | 8.48 | 0.43 | 177.26 | 173.62 | 2.07 | 0.64 |
| Std. Dev. | 21.42 | 20.23 | 2.38 | 0.02 | 17.45 | 17.57 | 0.39 | 0.05 |

The above clearly demonstrates that PCU implants of the current invention are significantly more durable than silicone elastomer in conditions of abrasive wear against a rough counter face which is the expected situation in vivo. More specifically, as shown in Table 2, the current silicone specimens wear 4 times more than the PCU implant specimens under uniform testing conditions for both groups.

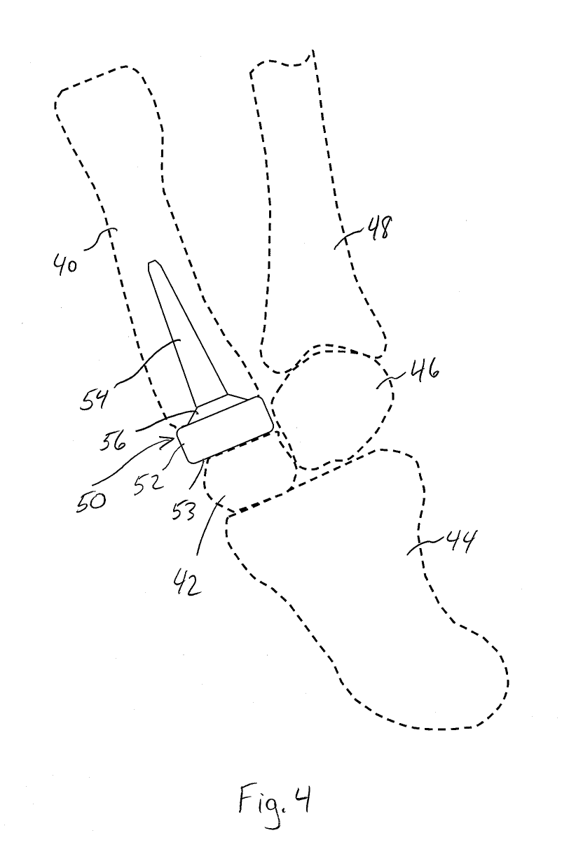

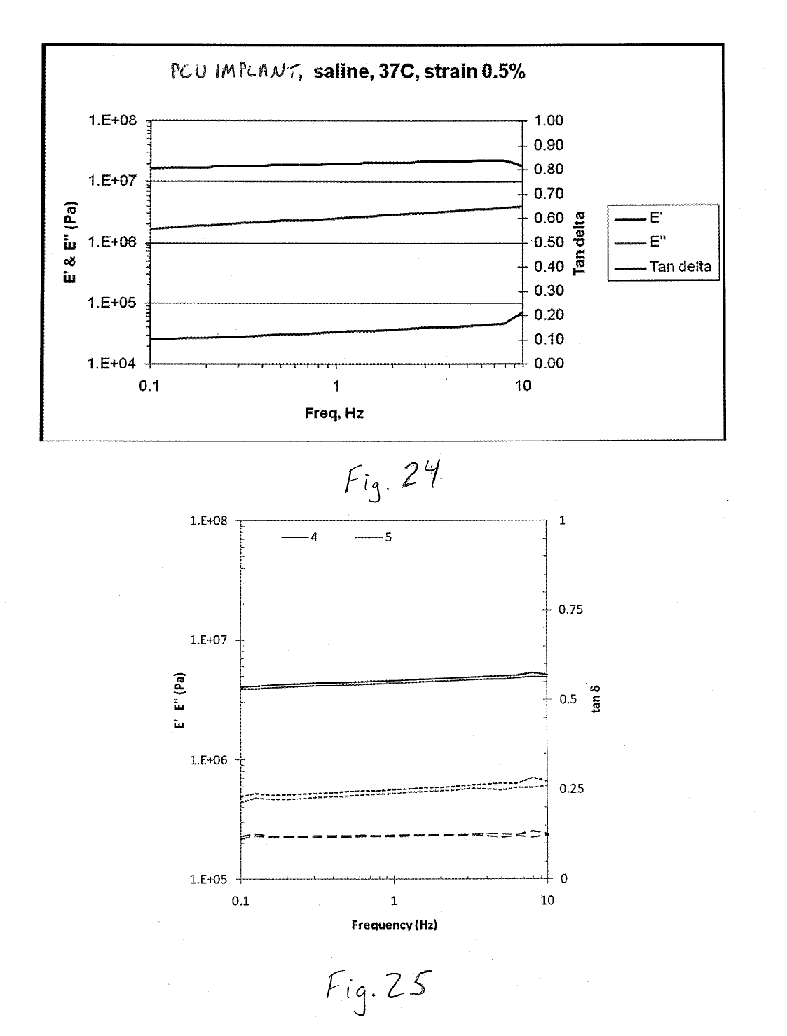

Furthermore, FIG. 23 shows a scanning electron microscope picture of the surface of an artificial bone 30 counter face that was pressed against the PCU implants, similar to FIG. 3 which shows the artificial bone 30 counter face that was pressed against the silicone implants. As seen in FIG. 23, the PCU implants did not have significant material transfer like the silicone and the pores 32 remain clear and there are no ridges formed.

It was clear from the wear tests that the PCU implant showed significantly less wear against an artificial bone counter face. Volumetric wear is significantly less and is demonstrated by significantly less weight loss from the PCU implant sample when compared to that of the silicone elastomer implant.

In light of the fact that there is less volumetric wear of the PCU implants, and no electron microscopic evidence evidence for transfer wear as demonstrated by the scanning electron microscopy, it is believed that particulate synovitis can be avoided with the use of a more biomechanically and biomaterially sound elastomeric implant material of the present invention.

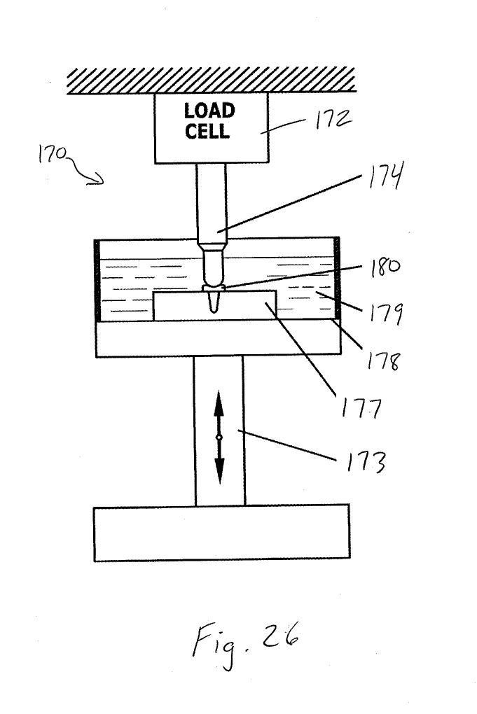

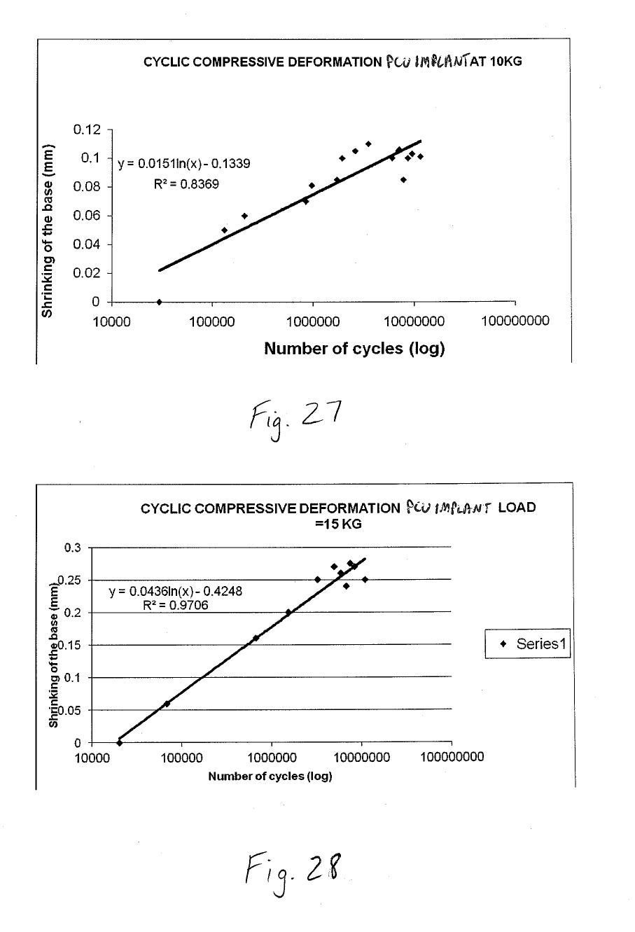

To further confirm the viability of the implants of the present invention, a thermal dynamic mechanical analysis of the silicone elastomer and the PCU implant samples were carried out at 37° C. and the results are charted in FIGS. 24 and 25. The results show that the PCU implant samples are about 5 times more stiff in compression than silicone elastomer in vivo. The stiffness of the silicone samples at 37° C. under dynamic compression at 0.5% strain is approximately 4 Megapascals, whereas on the other hand the stiffness of the PCU implant samples are at approximately 20 megapascals.

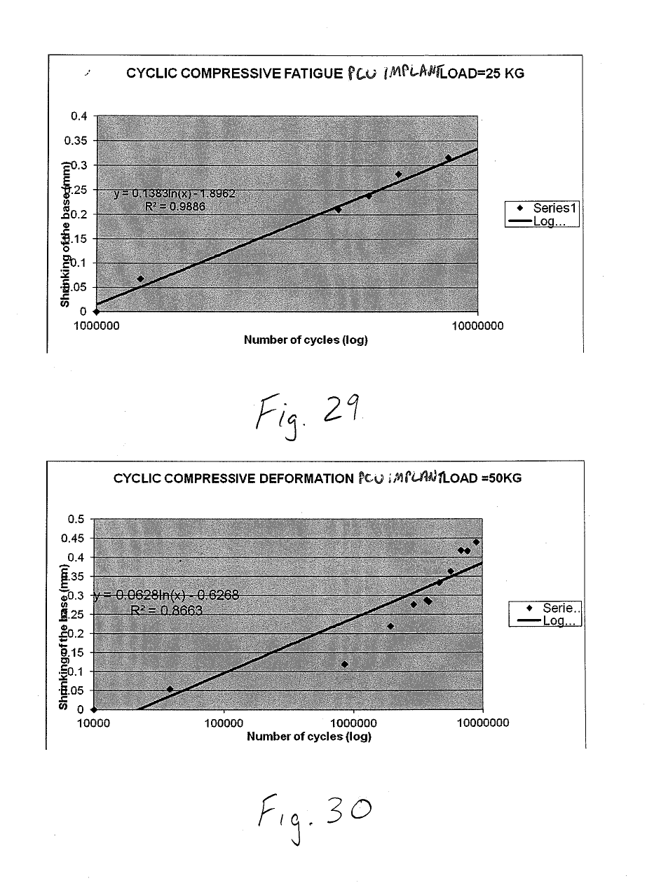

As a further confirmation, the PCU implants specimens were subjected to a cyclic compressive fatigue test using a fatigue testing assembly 170 as shown in FIG. 26. The assembly 170 was an Instron testing machine (Model of machine—8500.) with a small capacity load cell (3 Kip) 172 with a stainless steel rod 174 depending therefrom.. The specimen 180 was supported beneath the rod 174 in an implant holder 177 which was submerged in a saline 179 at 37° C. within chamber 178. The specimen 180 was equilibrated in the saline 179 for two days prior to the fatigue cyclic compression test.

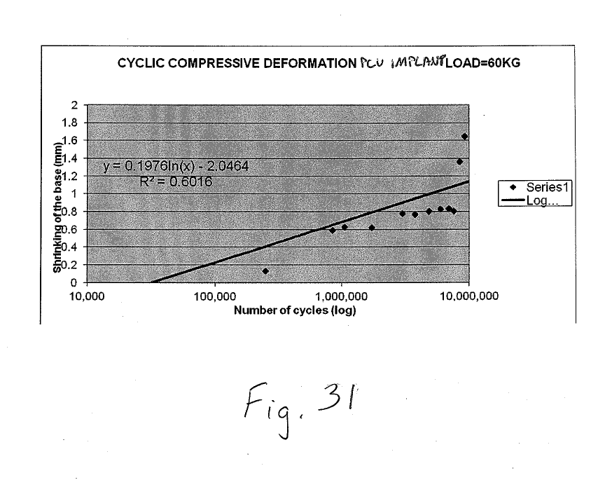

The assembly 170 was on the LOAD control, half sine wave form (sine wave, only compression force−half sine). For example—the system was run from minus 0.5 Kg to minus 60 Kg. Frequency was set at 10 Hz. For stability of the wave form and force we used a special mode of amplitude control. Five different loads were tested at 10 kg, 15 kg, 25 kg, 50 kg, and 60 kg. At each load the testing took approximately 14 days to achieve 10 million cycles of compressive fatigue. As shown in FIGS. 27-31, the PCU implant remained structurally stable to 10 million cycles at all five loads tested.

What is claimed is:

1. A compressive force and compressive-shear force joint implant, comprising:
a head defining at least one wear contact surface wherein at least each wear contact surface is manufactured from a polycarbonate urethane material.

2. The implant of claim 1 wherein the head has a cylindrical configuration with opposed wear contact surfaces at the opposed flat ends of the cylinder.

3. The implant of claim 2 wherein the entire head is manufactured from the polycarbonate urethane material.

5. The implant of claim 1 wherein the head has a spherical configuration and defines wear contact surfaces at least two distinct areas of the surface of the sphere.

6. The implant of claim 5 wherein the entire head is manufactured from the polycarbonate urethane material.

7. The implant of claim 1 further comprising a stem extending from the head opposite of the at least one wear contact surface.

8. The implant of claim 7 wherein the head and stem are a unitary structure manufactured from the polycarbonate urethane material.

9. The implant of claim 1 wherein the head defines at least one through passage extending therethrough in a plane substantially parallel to the at least one wear contact surface.

10. A method of performing a CMC arthroplasty on a subject, comprising the steps of:
removing a portion of a trapezium of the subject; and
positioning an implant in accordance with claim 1 between a remaining portion of the trapezium and an adjacent metacarpal of the subject such that the at least one wear contact surface is in compressive contact with the remaining portion of the trapezium.

11. A method according to claim 10 comprising the step of shaping one or both of the trapezium and metacarpal prior to insertion of the implant, and wherein the implant is positioned such that a second wear contact surface is in compressive contact with the metacarpal.

12. A method according to claim 10 comprising the step of forming a bore in the metacarpal prior to insertion of the implant, and wherein the step of positioning the implant includes positioning a stem extending from the head opposite the at least one wear surface into the bore.

13. A method of performing a CMC arthroplasty on a subject, comprising the steps of:
removing a trapezium of the subject; and
positioning an implant in accordance with claim 1 between a scaphoid of the subject and an adjacent metacarpal of the subject such that the at least one wear contact surface is in compressive contact with the scaphoid.

14. A method of performing a CMC arthroplasty on a subject, comprising the steps of:
removing a trapezium of the subject;
positioning a first implant in accordance with claim 1 relative to a scaphoid of the subject such that the at least one wear surface of the first implant faces away from the scaphoid; and
positioning a second implant in accordance with claim 1 relative to a metacarpal of the subject such that the at least one wear contact surface faces away from the metacarpal and is in compressive contact with the at least one wear surface of the first implant.

15. A method of performing a MTPJ arthroplasty on a subject, comprising the steps of:
forming a bore in a proximal phalange of the subject; and
positioning an implant in accordance with claim 1 between the proximal phalange and an adjacent metatarsal of the subject such that a stem extending from the head opposite the at least one wear contact surface is received in the bore and the at least one wear contact surface is in compressive contact with the metatarsal.

16. A method of performing a radiocapitellar joint arthroplasty on a subject, comprising the steps of:
forming a bore in a radius of the subject; and
positioning an implant in accordance with claim 1 between the radius and an adjacent humerus of the subject such that a stem extending from the head opposite the at least one wear contact surface is received in the bore and the at least one wear contact surface is in compressive contact with the humerus.

17. A compressive force and compressive-shear force joint implant, comprising:
a head defining a wear contact surface, and
a stem extending from the head opposite of the wear contact surface,
wherein at least the wear contact surface is manufactured from a polycarbonate urethane material.

18. The implant of claim 17 wherein the entire head is manufactured from the polycarbonate urethane material.

19. The implant of claim 18 wherein the head and stem are a unitary structure manufactured from the polycarbonate urethane material.

20. The implant of claim 17 wherein the head defines at least one through passage extending therethrough in a plane substantially parallel to the wear contact surface.
